(19) United States
(12) Patent Application Publication
(10) Pub. No.: US 2022/0254451 A1
(43) Pub. Date: Aug. 11, 2022

(54) Nucleic Acid-Based Data Storage

(71) Applicant: Biosistemika D.O.O., Ljubljana (SI)

(72) Inventors: Klemen Zupancic, Ljubljana (SI); Nejc Bernot, Ljubljana (SI); Zan Pirc, Ljubljana (SI); Jasna Lalic, Zapresic (HR); Kristina Gruden, Ljubljana (SI); Urska Cepin, Ljubecna (SI); Blaz Nemec, Rence (SI); Luka Zupancic, Ljubljana (SI)

(21) Appl. No.: 17/291,664

(22) PCT Filed: Nov. 7, 2019
(86) PCT No.: PCT/EP2019/080592

§ 371 Date: May 6, 2021

(30) Foreign Application Priority Data
Nov. 7, 2018 (EP) 18205046.8
May 29, 2019 (EP) 19177466.0

(51) Int. Cl. G16B 50/20; G06N 3/12; G11C 11/54

(52) U.S. Cl. CPC G16B 50/20; G11C 11/54; G06N 3/123

(57) ABSTRACT

A method of storing information in nucleic acid includes processing units of information into permutation numbers by a reversible algorithm, providing a library of n distinct oligonucleotide strings of predetermined length in a fixed order, n being a positive integer and each distinct oligonucleotide string associated with a distinct index indicating the ordinal position, assembling distinct oligonucleotide strings to create strands comprising at least two oligonucleotide strings, each oligonucleotide string's ordinal position matching with a permutation number, each strand including at least a data bearing part and a semantic part, and the semantic part to allocate orientation and/or order to a strand.

### FIELD OF THE INVENTION

The present invention is concerned with a method of storing information in nucleic acids. The method comprises encoding digital data into sequences of nucleotides. These sequences are then assembled, for example enzymatically via PCR amplification. The sequences comprise the digital information and can be decoded by sequencing followed by using the encoding parameters to decode the sequence into the initial digital sequence.

### BACKGROUND

During the last several years a new era called the “data age” has arisen. The data age is characterized by the quick transition of analog to digital data, alongside with a huge increase in new data being generated on daily basis. Data has become critical to all aspects of life with the rise of internet; smart home devices and internet of things, communication and social media, autonomous cars, humanoid robots, etc. transforming deeply many aspects of human life. This digital existence, as defined by the sum of all data created, captured, and replicated on earth in any given year is growing rapidly.

The amount of digital data in the world is exponentially growing, but ability to store all that data is not keeping pace. It is expected that by 2025 the “global datasphere” will grow to 163 zettabytes (that is a trillion gigabytes), that is ten times of data generated in 2016. Current infrastructure can handle only a fraction of the coming data deluge, which is expected to consume all of the world's microchip-grade silicon by 2040. This fundamental change gives rise to the new challenges of managing, interpreting and storing big data.

Another problem is the lifetime of the presently used standard media for archiving data, such as optical discs, hard drives, and magnetic tapes, which lifetime is only a few years.

Therefore there is a need for an alternative storage medium, which stores data efficiently and reliably for an extended period of time.

DNA appears to be an excellent candidate for an alternative storage medium due to its enormous information capacity, extreme spatial compactness, long term stability and basically no maintenance costs. All living organisms run on the same software language: DNA. In other words DNA has proven to be a stable, robust and long-living medium. There are two main approaches to DNA synthesis: chemical, using phosphoramidite synthesis and enzymatic, using template-free polymerase (terminal deoxynucleotidyl transferase). The former uses solid state whereas the latter is an aqueous process.

There have been several attempts to use DNA for storing data, with approaches based on either chemical or enzymatic synthesis of DNA.

Examples for an approach using chemical synthesis is semiconductor-based synthetic DNA manufacturing process featuring a high-throughput silicon platform with parallel synthesis, another one is generation of large quantities of a few different DNA molecules with up to about 30 base pairs and using combinatorial enzymatic reactions to encode information into the recombination patterns of those prefabricated bits of DNA. In the latter, instead of mapping one bit to one base pair, bits can be arranged in multidimensional matrices, and sets of molecules represent their locations in each matrix.

Examples for an approach using enzymatic synthesis of DNA is a three-step enzymatic DNA synthesis by using terminal deoxynucleotidyl transferase (TdT) and a reversible terminator.

While progress is made in developing methods for storing information into DNA there still is an urgent need for an improved, cost-efficient, and reliable method of storing information in DNA due to the increasing data creation and the consequent increasing need for data storage.

### DESCRIPTION OF THE INVENTION

The present invention provides an improved, cost-efficient, and reliable method of storing information in DNA. The invention combines a novel and inventive encoding algorithm with enzymatic synthesis of polynucleotides. The present invention is based on custom combination of precast DNA pieces into larger assemblies which comprise the digital information and can be decoded by sequencing using the encoding parameters to decode DNA sequence into the initial digital sequence. Those assemblies comprise non-constant information and mutually semantically overlapping parts so the encoding/decoding order is known.

### Definitions

The following terms are used in this description:

A “string” is an oligonucleotide, i.e. a piece of DNA sequence that comprises coding information and optionally a gluing part. Strings are combined to form ropes and/or braids. The length of a string can be from a few bases such as three or four bases up to hundreds or thousands bases. A string can be single-stranded (ss-string) or double-stranded (ds-string). The term string comprises single and double stranded sequences.

A “rope” comprises two strings and optionally a gluing part between the two strings. A rope can be single-stranded (ss-rope) or double-stranded (ds-rope). The term rope comprises single and double stranded sequences.

A “braid” is a combination of strings and/or ropes. In other words, at least three strings can form a braid. The number of strings in a braid is variable, it can be only a few such as three to six, or medium size, such as 10 to 500, or big size, such as 500 to 5000, or mega size, such as 5000 to many thousand, for example 5 million. The size of a braid defines the amount of information and, thus the size depends on the information to be stored. The braid can be built from strings or from ropes and can optionally comprise gluing parts. A braid has a specific structure, it comprises at least a head string and a tail string, and usually at least two inner strings, between the head string and the tail string.

A “gluing part” is a sequence that does not contribute information but forms an overlapping end to allow combining two strings in a predetermined order to form a rope. The gluing part can have any length that allows combining, such as about 3 to about 20 bases. The gluing part can have any sequence that allows combining and does not interfere with the coding part, i.e. the strings. Gluing parts of different strings can be different or equal, preferably the gluing sequence is the same for all strings.

A “data unit” is a group of braids carrying the information to be stored. A data unit comprises a “first braid”, an even number of “head/tail braids” and a “terminal braid”, wherein in the first braid the head string is a starter sequence, that is a unique string that is only present in the first braid and nowhere else in any other braid and/or within the same braid. Wherein the inner part of a braid comprises a number of strings and a tail string, a number of braids that comprise a head string, an inner part comprising a number of strings and a tail string, wherein the tail string is a terminal string that is a unique tail string and is present only once as a tail string in a data unit. By this arrangement of strings in braids, the order of braids is defined so that a data unit can be decoded without problems.

“Information to be stored” can be any information that is present in a form that can be translated in a code, in particular binary data, such as data pools, databases, research data, books, pictures, movies, etc.

A “string library” is a library of groups of strings, wherein any string has the same number of bases and wherein in a group each string has the same sequence, and strings of different groups have different sequences, wherein a string of one group cannot have the same sequence as a string of another group.

A “rope library” is a library of groups of ropes, wherein any rope comprises two different strings and wherein in a group each rope has the same sequence, and ropes of different groups have different sequences, wherein a rope of one group cannot have the same sequence as a rope of another group.

The method of the present invention allows to assemble DNA carrying information from smaller units, i.e. strings or ropes, in a very efficient, resource-saving way. The method of the present invention is as defined in the claims.

In short, information, such as a binary code, is translated into pieces of nucleic acids which have a structure that allow easy coding and decoding. The method of the present invention and the structure of the DNA parts used provide a high flexibility and allow to choose and adapt elements for any type of data, and for any amount of information.

The number of bases in a string defines the number of available permutations, for example when strings with a length of 4 bases are used, 256 permutations are available. Each group of strings comprises only one type of permutation. The terms “string” and “permutation” can be used interchangeably. The number of permutations defines the number of braids available for one data unit. If the number of permutations is n, the number of available braids is n−1. Thus, the length of the strings and the number of braids can be chosen depending on the amount of information to be stored in a data unit.

To decode the information of a data unit it is necessary to know the order of the braids and, thus the position of each braid in a row, i.e. if a braid is the first one, the second one, the n^{th} one or the terminal one. The present invention provides a structure that allows this allocation. In the braids, the heads and tails provide the information about the “position” of a braid, because the head string of a braid is identical to the tail string of the preceding braid, starting string and terminal string are unique. For a data unit, there will be one starting string, one terminal string and pairs of head and tail strings where each pair can be used only once as end string in a braid of a data unit.

The method of the present invention uses a library which can be a string library or a rope library. The library comprises a number of strings of identical length or a number of ropes of identical length.

In one approach in a first step two strings are combined to create ropes, in a second step ropes are assembled to create braids. In another approach, a library of ropes is created by combining strings and the ropes of the library are assembled to create braids.

Any string is an oligonucleotide comprising a coding part that carries the information and optionally a gluing part that can be used for combining two strings to form a rope. The ropes are to braids. The information to be stored is contained in a group of braids that is designated as data unit.

In one embodiment the method of storing information in nucleic acid comprises providing a library comprising a plurality of types of single-stranded oligonucleotides, which are also called “strings”. The strings each comprise a unique coding nucleotide sequence. Each type of string is provided in a separate vessel. The plurality is defined as being x, wherein x is an integer. For example the index can be an integer from 0 to x−1, each index being assigned to one type of strings, such as single-stranded oligonucleotides. The value of x is dependent on the number of bits to be encoded and the number of nucleotides in a string. The number of nucleotides in a string defines the number of permuations available. As an example, if the strings have 4 nucleotides, 16 permutations are possible and because of restrictions there are 14 different strings in the library, the strings will be assigned an index starting with 0 and going to 13. The strings are the coding blocks which are available to encode information from binary form into a sequence of nucleotides.

The method of the present invention allows to store information in binary form as nucleic acid. The translation of information from binary form to DNA form can be done directly or via a transcription, for example by using a decimal form. Any algorithm providing the translation and/or transcription can be used. A particulary useful method is outlined below. Methods of converting information such as text strings or image data into binary form are known in the art. An ASCII text can for example be converted into binary with 7 bit-per-letter encoding resulting in the binary sequence, which is then to be encoded into a nucleotide sequence.

The method further comprises encoding the information in binary form into the nucleotide sequence of a plurality of polynucleotides, which are also called “braids”. Each braid comprises an ordered linear arrangement of strings selected from the library, wherein the ordered linear arrangement determines the linear arrangement of a plurality of strings combined in each braid. The string in the first position is the string that is the first sequence starting from the 5′ end of the polynucleotide. Each braid comprises the same number of strings. The number of strings within a braid is variable and can be chosen depending on the length of a string, the amount of information to be stored etc.

The maximal number of braids that can be used per data unit is the number of strings/permutation within the library minus 1. If for example a library comprises 6 different strings, then up to 5 different braids can be made. There are three types of braids, wherein one type of braids is the first or starting braid, one type of braids is the last or terminal braid, and one type of braids is a middle or internal braid.

The single-stranded oligonucleotide in the first position starting from the 5′ end in the first polynucleotide, which is also called the first string of the first braid, is different from the first string in any one of the other types of braids. Therefore the first braid is identifiable from the other braids by its unique starting string. The single-stranded oligonucleotide in the last position starting from the 5′ end, which is also called the last string of the braid, is the same as the first string in a second braid. Therefore internal braids are identifiable by having first and last strings which correspond to first and last strings of a further braid. The order of the internal strings is identifiable by matching the last string of a braid to the first string of the subsequent braid. The last string of the last braid of the plurality of braids is different from the first string in any one of the other types of braids. Therefore the terminal braid is identifiable by its unique last string, and by a first string which matches the last string of the last internal braid. The first string in each braid is unique. Furthermore, each string only occurs once per braid.

The present invention also provides an encoding algorithm that is outlined below and that complies with the above mentioned parameters, and will define the ordered linear arrangement in all braids via calculation of partial permutations (see the proof of concept in the Example).

Although the strings forming the braids can be assembled by any known method for assembling oligonucleotides, the present invention provides an encoding algorithm that is particularly useful and assembles the strings via enzymatic DNA synthesis using the single-stranded polynucleotides as primers. This specific method has the advantage that it is “green technology” avoiding or minimizing the use of chemical agents.

In one embodiment the braids are assembled via an intermediate amplification step resulting in oligonucleotides comprising two unique coding sequences. In this embodiment of the method of the invention the library of single-stranded oligonucleotide comprises a plurality of pairs of single-stranded oligonucleotides, wherein each pair comprises a forward and a reverse string or primer, wherein the forward string comprises a first single-stranded oligonucleotide comprising a first coding sequence at its 5′ end and a first gluing sequence at its 3′ end, and wherein the reverse string or primer comprises a second coding sequence at its 5′ end and a second gluing sequence at its 3′ end, wherein the second coding sequence is complementary and inverse to the first coding sequence, and wherein the second gluing sequence is complementary and inverse to the first gluing sequence, and wherein the first gluing sequence in each pair of single-stranded oligonucleotides is identical, and wherein each coding sequence is unique.

As the strings are used as primers in a PCR, the sequence of the strings need to comply with the requirements for primers as is known in the art. This refers for example to G/C content, length, and to avoiding secondary structures, such as hairpins or loops. The G/C content can for example be between 0.5 and 0.55. The coding sequence of the single-stranded oligonucleotide can for example have a length of about 3 to about 500 nucleotides, such as about 6 to about 100 nucleotides, for example 8 to 20 nucleotides. As outlined before the number of nucleotides defines the number of permutations and, thus, the amount of information to be stored. The smaller the oligonucleotides the more stable are the strings but the fewer the number of permutations. The higher the number of nucleotides in a string the higher the number of permutations but the higher the risk for errors.

It has been found that good results are obtained when exactly two G/C bases are present in the last 5 bases of each coding sequence. Furthermore, it has been found that it is useful that there are no more than two identical bases in a row, and no more than two of such 2-base repeats are present in each coding sequence. this avoids problems with secondary structures that disturb the assembly.

Furthermore the Levenshtein distance between each pair of coding sequences should sufficient to to make it possible to detect and correct errors. The Levenshtein distance can be at least 5, for example. This means that at least 5 single base changes are required to turn one coding sequence into another coding sequence. If there are no more than 2 errors, the resulting sequence will still be closer to the correct original sequence that to any other of the coding sequences.

In this embodiment a series of first amplification reactions is carried out to obtain double-stranded oligonucleotides, which are also called “ropes”. Each rope comprises two unique coding sequences separated by the gluing sequence in the middle. The first coding sequence and the internal gluing sequence are derived from the first string used as a primer in the amplification reaction, the second coding sequence in the rope is obtained by extending the first string using the complementary and inverse sequence of a second coding sequence as a template.

A rope can for example be obtained by annealing a forward primer of a first pair of single-stranded oligonucleotides to a reverse primer of second pair of single-stranded oligonucleotides, extending both the reverse and forward primers by PCR to obtain the double-stranded oligonucleotide. One strand of the rope comprises the coding sequence of the forward primer of the first pair at its 5′ end, and the coding sequence of the forward primer of the second pair at its 3′ end, wherein the gluing sequence is between the coding sequences., wherein the first and second pair are selected based on the order determined in step c) of claim 1. The gluing sequence can have has a length of about 8 nt.

The amplification reaction resulting in the rope formation is carried out for each combination of strings necessary to assemble the braids as defined by the encoding algorithm. If a braid has been defined as having the order of strings 4-1-13-12-8-10, for example, then the following ropes have to be obtained: 4-1; 1-13, 13-12, 12-8, and 8-10.

Therefore the rope amplification is repeated with all combinations of primer pairs until all combinations of double-stranded oligonucleotides present in the order determined in the encoding algorithm have been obtained.

After all necessary ropes have been obtained, the braids are assembled by pooling the ropes that have to be present in one braid as determined by the encoding algorithm separately for each braid. The mixture of ropes is then amplified via PCR to obtain the completed braids. The ropes act as primers and templates for the polymerase. The end-standing ropes, i.e. the rope comprising the first string of the braid, and the rope comprising the last string of the braid should be overrepresented in the pool to facilitate the desired braid assembly. The amplification is carried out until the braid is complete. Intermediate products comprising only parts of the braid will be present in a small amount.

The completed braid can be purified by methods as known in the art to remove the intermediate products. This can for example be done by size-separation on a gel, followed by extraction from the gel as is known in the art.

In a further embodiment the amplification reaction creating the ropes is skipped. This can be done by designing a library of single-stranded oligonucleotides comprising a plurality of single-stranded oligonucleotides, each comprising two unique coding sequences derived from any combination of single-stranded oligonucleotides comprising one coding sequence.

These single-stranded oligonucleotides are similar to the strands of the ropes as defined above, but do not carry an internal gluing sequence. With these types of oligonucleotides comprising two coding sequences it is possible to assemble the braids in one PCR amplification step instead of two PCR amplification steps by pooling the single-stranded oligonucleotides each comprising two coding sequences present in each polynucleotide separately for each polynucleotide, and amplifying the mixture of oligonucleotides to obtain the completed polynucleotides as described above.

The resulting braids can then be purified as described above for the two-step amplification method.

The present invention also comprises a method of accessing the data information stored in the nucleic acid according to the method of the invention by sequencing the polynucleotides and assembling the information based on the encoding information.

The amplification reactions used in the method of the present invention can be any amplification method suitable for the purpose of assembling ropes, or braids.

The same applies to the sequencing reactions used to retrieve to information stored in the DNA according to the present invention.

### BRIEF DESCRIPTION OF THE DRAWINGS

FIG. 1 shows the experimental design and the nomenclature used in the methods of the present invention. Step 1 shows the STRINGS, which are single-stranded oligonucleotides comprising a unique coding sequence and a gluing sequence (yellow). These strings are used as primers. Each string is present as forward primer and as reverse primer. Step 2 shows the rope formation using a pair of forward and reverse primers by PCR amplification. Two strings of choice (determined by the encoding order set by the encoding algorithm) are annealed via the gluing sequence, elongated and PCRed to give ROPES, each of which consists of two coding sequences. Step 3 shows the braid formation. BRAIDS are encoding/decoding units that consist of a number of coding sequences (5 coding sequences in the example shown in FIG. 1). The braids are obtained by mixing equivolume amount of ropes and subjecting them to PCR using an excess (e.g. 10 times more) of the end ropes as primers.

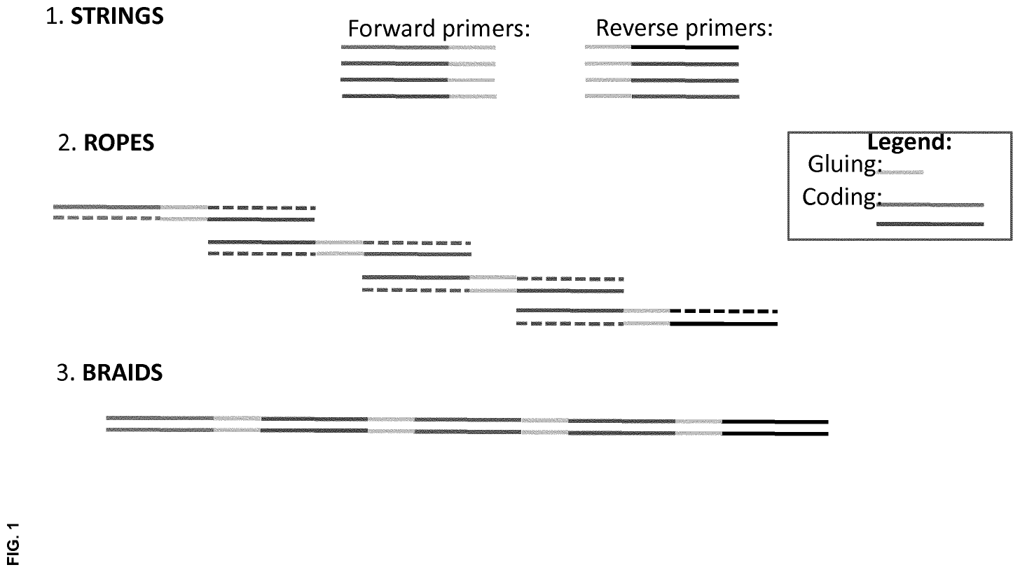

FIG. 2 shows a gel image of the Ropes in which the word “BIOSISTEMIKA” is to be encoded. Four μl of each PCR product is loaded. On the left-hand side to the gel image, the O'GeneRuler Ultra Low Range DNA Ladder is denoted. Starting from left to right, the first lane contains 200 ng of the DNA ladder and the second lane contains-control 44 bp Rope DNA product followed by first 17 Ropes (FIG. 2A) denoted in the Table 3, and followed by the last eight Ropes denoted in the Table 3 (FIG. 2B).

FIG. 3 shows a gel image of the five Braids in which “BIOSISTEMIKA” is encoded. Four μl of each PCR product is loaded. On the left-hand side to the gel image, the O'GeneRuler Ultra Low Range DNA Ladder is denoted. Starting from left to right, the first lane contains 200 ng of the DNA ladder and the second lane contains quasi-control 154 bp DNA product followed by five Braids: 4 1 13 12 8 10, 10 11 3 1 0 7, 7 3 11 10 4 0, 0 5 6 3 1 15 9 and 9 4 13 2 3 5 and 750 ng of Quick-Load® 100 bp DNA Ladder and 200 ng of O'GeneRuler Ultra Low Range DNA Ladder.

In the following a method for preparing a coding data unit is outlined. This is only examplary to more clearly show how ropes and braids can be obtained and how a data unit can be assembled.

If there are not enough bits that need to be encoded in the middle of a group the group is padded by filling them with zeroes. For example if 8 bits are encoded in the current braid, but it is only necessary to encode two bits (11 for example), 11000000 will be encoded. The number of ‘padding’ bits will need to be stored as part of the file metadata (together with the DNA storage location etc).

The decoding process works in the same way, just in reverse. The detected permutation is enumerated, the number of available permutations counted and then the enumeration converted to the correct number of bits.

A 2 letter redundancy is used meaning that up to two errors in the read coding sequences can detected and corrected. This is achieved by using a Levenshtein distance of 5, meaning that at least 5 (single base) changes are required to turn a used coding sequence into another used coding sequence. If there are no more than 2 errors, the resulting sequence will still be closer to its original state than any other used coding sequence.

### Partial Permutation Enumeration

Throughout the text the term ‘enumerating’ partial permutation is used. This means that a number is assigned to each possible partial permutation, in a certain order.

The system of enumerating them is similar to a regular numerical system, but instead of the factors that are multiplied the digs with being powers of the base of the numerical system, they are the partial permutation numbers of the available symbols.

The following is an example for enumeration:

A certain number of symbols is available (n). These symbols must be in an ordered list.

A certain partial permutation of those symbols (m symbols) is chosen. This is done by choosing the first symbol and removing it from the list (since symbols cannot repeat in a permutation). The second symbol is then chosen from this now shortened list and so on. This is repeated until m symbols have been chosen.

The index (in the remaining list) of the symbol is marked and chosen at each step with oi. The index starts with 0.

The enumeration of the partial permutation obtained this way is:

$${\sum }_{i = 1}^{m} ( {o}_{i} * {\prod }_{j = n - m + 1}^{n - i} j )$$

It is assumed that the following symbols are available: [0,1,2,3,4,5,6,7,8,9]

This results in the following partial permutation: 1 0 4

a) First Symbol

1 is the second symbol in the available list, giving an o1 of 1.

The product results in 72 (9*8).

This means that this symbol supplies the value of 72 (72*1)

b) Second Symbol

0 is the first symbol in the available list, giving an o2 of 0.

The product results in 8.

This means that this symbol supplies the value of 0 (8*0)

c) Third Symbol

4 is the third symbol in the available list ([2,3,4,5,6,7,8,9]), giving an o3 of 2.

The product results in 1.

This means that this symbol supplies the value of 2 (1*2)

The sum of these values gives the partial permutation enumeration of 74.

The invention is in the following further explained by describing some examples.

### EXAMPLES

### Example 1—Proof of Concept Encoding the Word “BIOSISTEMIKA”

In a first step a set of DNA sequences that were used as strings in the encoding system of the present invention was defined. The strings were chosen in a way that fulfilled a number of conditions:

a) Sufficient Levenshtein distance between each pair of sequences, making it possible to detect and correct errors

b) A GC content between 0.5 and 0.55

c) Exactly two G/C bases in the last 5 bases

d) No more than two identical bases in a row, and no more than 2 of such repeats in the sequence.

In this example, sequences with a length of 18 nucleotides were used. To encode the word “BIOSISTEMIKA” in DNA, a set of 14 forward and 14 reverse single-stranded strings (standard desalting purification) were designed as set forth in Table 1. The string sequences should satisfy the premises described above.

TABLE 1
Set of strings used for encoding “BIOSISTEMIKA” in DNA. Coding part is
denoted in capital letters whereas gluing part in denoted in lowercase.

| Name | Forward ss-string sequence | Name | Reverse ss-string sequence |
|---|---|---|---|
| C8G 0F | GCAATTGATGGCGGTAGAgcgacaga | C8G 0R | TCTACCGCCATCAATTGCtctgtcgc |
| | SEQ ID NO: 1 | | SEQ ID NO: 2 |
| C8G 1F | TCTGCAAGCTGGAGTAGAgcgacaga | C8G 1R | TCTACTCCAGCTTGCAGAtctgtcgc |
| | SEQ ID NO: 3 | | SEQ ID NO: 4 |
| C8G 2F | TAGGATACGCCACACACTgcgacaga | C8G 2R | AGTGTGTGGCGTATCCTAtctgtcgc |
| | SEQ ID NO: 5 | | SEQ ID NO: 6 |
| C8G 3F | TAACGTCGGCTACTCACAgcgacaga | C8G 3R | TGTGAGTAGCCGACGTTAtctgtcgc |
| | SEQ ID NO: 7 | | SEQ ID NO: 8 |
| C8G 4F | TCGTGTTAGTCCGTCAGTgcgacaga | C8G 4R | ACTGACGGACTAACACGAtctgtcgc |
| | SEQ ID NO: 9 | | SEQ ID NO: 10 |
| C8G 5F | AACACTCGTCTCGACCATgcgacaga | C8G 5R | ATGGTCGAGACGAGTGTTtctgtcgc |
| | SEQ ID NO: 11 | | SEQ ID NO: 12 |
| C8G 6F | TCTATGCGACCACTACGAgcgacaga | C8G 6R | TCGTAGTGGTCGCATAGAtctgtcgc |
| | SEQ ID NO: 13 | | SEQ ID NO: 14 |
| C8G 7F | TGACAGAGCGTCTATCGTgcgacaga | C8G 7R | ACGATAGACGCTCTGTCAtctgtcgc |
| | SEQ ID NO: 15 | | SEQ ID NO: 16 |
| C8G 8F | ATGGCTATCGCTGATTGCgcgacaga | C8G 8R | GCAATCAGCGATAGCCATtctgtcgc |
| | SEQ ID NO: 17 | | SEQ ID NO: 18 |
| C8G 9F | TCCTGCGCTTATCAGAGTgcgacaga | C8G 9R | ACTCTGATAAGCGCAGGAtctgtcgc |
| | SEQ ID NO: 19 | | SEQ ID NO: 20 |
| C8G 10F | TCGCTAGAAGAGCAGAGTgcgacaga | C8G 10R | ACTCTGCTCTTCTAGCGAtctgtcgc |
| | SEQ ID NO: 21 | | SEQ ID NO: 22 |
| C8G 11F | AGTGTCCAGGATTGCATGgcgacaga | C8G 11R | CATGCAATCCTGGACACTtctgtcgc |
| | SEQ ID NO: 23 | | SEQ ID NO: 24 |
| C8G 12F | ACCGATACACGTCGTACAgcgacaga | C8G 12R | TGTACGACGTGTATCGGTtctgtcgc |
| | SEQ ID NO: 25 | | SEQ ID NO: 26 |
| C8G 13F | GTGCATGCATCACGATGAgcgacaga | C8G 13R | TCATCGTGATGCATGCACtctgtcgc |
| | SEQ ID NO: 27 | | SEQ ID NO: 28 |

In a second step the ASCII text “BIOSISTEMIKA” is converted into binary with 7 bit-per-letter encoding resulting in the following binary sequence:

100001010010011001111101001110010011010011101010010001011001101100100 110010111000001

The starting binary has a length 84 bits.

To convert from a number to a permutation, the permutations have to be enumerated. This is done by assigning a value to the ‘offset’ at each ‘spot’ of the permutation (the first choice is the first spot). The offset is the index of the choice that is made—picking the first choice that is available means an offset of 0, the second choice an offset of 1. The value of the offset is the number of possible partial permutations of all subsequent spots.

Encoding 3 bits (100, 4), with options 0 1 2 3 4 5 6 7 8 9 10 11 12 13 and 1 places, which encodes to 4

Here there are enough options to encode the first 3 bits. This is because there are 14 options (number of different strings), which is below 16 (2A4), but above (29). The value of these bits when converted to decimal is 4. Since this is encoded in only one ‘string’, the 5th option available (remember, 0 would be a valid option) has to be picked. Therefore the string with the assigned index “4” is picked.

Encoding 14 bits (00101001001100, 2636), with options 0 1 2 3 5 6 7 8 9 10 11 12 13 and 4 places, which encodes to 1 13 12 8

Here the situation is more complex, since the data in a sequence of 4 strings in encoded. There are a few more options (13 for the first spot, 12 for the second . . . , 13*12*11*10 in total, which is 17160, which translates into 14 bits (above 16384, but below 32768).

The enumeration of a permutation is calculated as follows. The 4th spot in the permutation will have a total of 10 options, and the value of an offset here will be 1. The 3rd spot will have 11 options, with the value of an offset here 10.

The 2nd spot will have 12 options, with the value of an offset here 110 (11*10).

The 1st spot will have 13 options, with the value of an offset here 1320 (12*11*10).

The value 2636 has to be converted into a linear combination of these values, which turns out to be 1320*1+110*11+10*10+1*6

The offsets are: 1, 11, 10, 6. The offset value determines, which of the available options for strings is picked. As the first option initially had the index 0, the offset value +1 is the number of the choice.

So for the first sequence the second one (offset value 1+1=2) of the available ones is picked, which is the string with the assigned index 1.

For the second sequence the 12th choice is picked. As 1 is no longer available here, the string with the assigned index 13 is picked.

For the third sequence the 11th choice is picked. 1 and 13 are no longer available, the string with the assigned index 12 is picked

And for the fourth sequence the 7th choice is picked, which is the string with the assigned index 8.

Therefore the first braid is composed of the following internal strings:

1-13-12-8

This way of encoding is the same for the center of all the other braids. There is a special situation that arises for the endings though—their options are not just restricted by the strings used inside their own strings, but also by the endings of all other braids (and the beginning of the first one). The encoding of the end is ‘increased’ by a value of 1 for each other end that has already been used and could still be generated by the options available and would have a value lower and equal than the value that we are trying to encode.

Encoding 2 bits (01, 1), with options 0 1 5 6 7 8 10 11 12 and 1 places, which encodes to 5

Normally, this would be encoded as 1 (the second option). However, since 0 has already been used as the ending of the third braid, the value to be encoded is increased to 2, which means we use the third option instead (5).

Therefore the encoding algorithm defines the braid compositions as follows. BIOSISTEMIKA=1000010100100110011111010011100100110100111010100100010110 01101100100110010111000001

### Start of First Braid

Encoding 3 bits (100, 4), with options 0 1 2 3 4 5 6 7 8 9 10 11 12 13 and 1 places, which encodes to 4

### Center of Braid 1

Encoding 14 bits (00101001001100, 2636), with options 0 1 2 3 5 6 7 8 9 10 11 12 13 and 4 places, which encodes to 1 13 12 8
   Ending of braid 1
Encoding 3 bits (111, 7), with options 0 2 3 5 6 7 9 10 11 and 1 places, which encodes to 10

### Center of Braid 2

Encoding 14 bits (11010011100100, 13540), with options 0 1 2 3 4 5 6 7 8 9 11 12 13 and 4 places, which encodes to 11 3 1 0

### Ending of Braid 2

Encoding 2 bits (11, 3), with options 2 4 5 6 7 8 9 12 13 and 1 places, which encodes to 7 Center of Braid 3
Encoding 14 bits (01001110101001, 5033), with options 0 1 2 3 4 5 6 8 9 10 11 12 13 and 4 places, which encodes to 3 11 10 4

### Ending of Braid 3

Encoding 2 bits (00, 0), with options 0 1 2 5 6 8 9 12 13 and 1 places, which encodes to 0 Center of Braid 4
Encoding 14 bits (01011001101100, 5740), with options 1 2 3 4 5 6 7 8 9 10 11 12 13 and 4 places, which encodes to 5 6 3 1

### Ending of Braid 4

Encoding 2 bits (10, 2), with options 2 4 7 8 9 10 11 12 13 and 1 places, which encodes to 9 Center of Braid 5
Encoding 14 bits (01100101110000, 6512), with options 0 1 2 3 4 5 6 7 8 10 11 12 13 and 4 places, which encodes to 4 13 2 3

### Ending of Braid 5

Encoding 2 bits (01, 1), with options 0 1 5 6 7 8 10 11 12 and 1 places, which encodes to 5

First gluing data: 3

Decoding braid 1

Decoding braid 2

Decoding braid 3

Decoding braid 4

Decoding braid 5

Final binary, length 84

Therefore BIOSISTEMIKA is encoded as:

4 1 13 12 8 10

10 11 3 1 0 7

7 3 11 10 4 0

0 5 6 3 1 9

9 4 13 2 3 5

Meaning that, for encoding “BIOSISTEMIKA” into DNA, 5 different braids are needed, each of which comprising 6 coding sequences (see Table 2).

TABLE 2
“BIOSISTEMIKA” encoding syntax: coding sequences
denoted as numbers which overlap between different
braids are denoted in bold.

| Braid annotation | Coding symbols present in |
|---|---|
| (by the order) | a particular braid |
| 1 | 4 1 13 12 8 10 |
| 2 | 10 11 3 1 0 7 |
| 3 | 7 3 11 10 4 0 |
| 4 | 0 5 6 3 1 9 |
| 5 | 9 4 13 2 3 5 |

The encoding algorithm provides the blueprint for the following assembly of the braids via PCR amplification.

Separate reactions were made by mixing forward and reverse primer pairs according to Table 3. Each reaction tube corresponds to a separate Rope.

TABLE 3
PCR reactions giving rise to the Ropes.

| | Rope | Forward | Reverse |
|---|---|---|---|
| Tube | annotation | ss-string | ss-string |
| 1 | 4-1 | C8G 4F | C8G 1R |
| 2 | 1-13 | C8G 1F | C8G 13R |
| 3 | 13-12 | C8G 13F | C8G 12R |
| 4 | 12-8 | C8G 12F | C8G 8R |
| 5 | 8-10 | C8G 8F | C8G 10R |
| 6 | 10-11 | C8G 10F | C8G 11R |
| 7 | 11-3 | C8G 11F | C8G 3R |
| 8 | 3-1 | C8G 3F | C8G 1R |
| 9 | 1-0 | C8G 1F | C8G 0R |
| 10 | 0-7 | C8G 0F | C8G 7R |
| 11 | 7-3 | C8G 7F | C8G 3R |
| 12 | 3-11 | C8G 3F | C8G 11R |
| 13 | 11-10 | C8G 11F | C8G 10R |
| 14 | 10-4 | C8G 10F | C8G 4R |
| 15 | 4-0 | C8G 4F | C8G 0R |
| 16 | 0-5 | C8G 0F | C8G 5R |
| 17 | 5-6 | C8G 5F | C8G 6R |
| 18 | 6-3 | C8G 6F | C8G 3R |
| 19 | 3-1 | C8G 3F | C8G 1R |
| 20 | 1-9 | C8G 1F | C8G 9R |
| 21 | 9-4 | C8G 9F | C8G 4R |
| 22 | 4-13 | C8G 4F | C8G 13R |
| 23 | 13-2 | C8G 13F | C8G 2R |
| 24 | 2-3 | C8G 2F | C8G 3R |
| 25 | 3-5 | C8G 3F | C8G 5R |

Each of the PCR reactions was performed in 20 μl volume using the following final concentrations: 200 μM of each of the dNTPs, 600 nM each of the forward and reverse strings and 1.2 U/100 μl reaction of Deep Vent DNA polymerase (NEB, 2U/μl). The thermal profile was: 30 sec at 30° C. (initial annealing), 1 min at 72° C. (initial elongation) followed by 20 cycles of 15 sec at 95° C., 15 sec at 30° C. and 15 sec at 72° C., ending with 5 min elongation step at 72° C.

The ideal thermal profile would imply using 28° C. in the annealing steps, since it is the theoretical annealing of the 8-nt long gluing sequence, but some thermal cyclers do not allow for this low temperature to be programmed during cycling. It was surprisingly realized that the assembly of ropes is not very susceptible to the annealing temperature because the ropes were created even when 50° C. annealing temperature was used. Using 50° C. annealing is recommended in this case since it is time-saving: it takes less time for the thermal cycler to heat or cool because the temperature difference between the PCR steps (95/72° C.) is smaller for 50° C. annealing in comparison to 30° C. annealing.

All the reactions were assembled on ice and quickly transferred to a thermocycler (Bio Rad T100) preheated to the denaturation temperature (95° C.). All components are mixed and centrifuged prior to use. It is important to add Deep Vent DNA polymerase last in order to prevent any degradation caused by its 3′→5′ exonuclease activity.

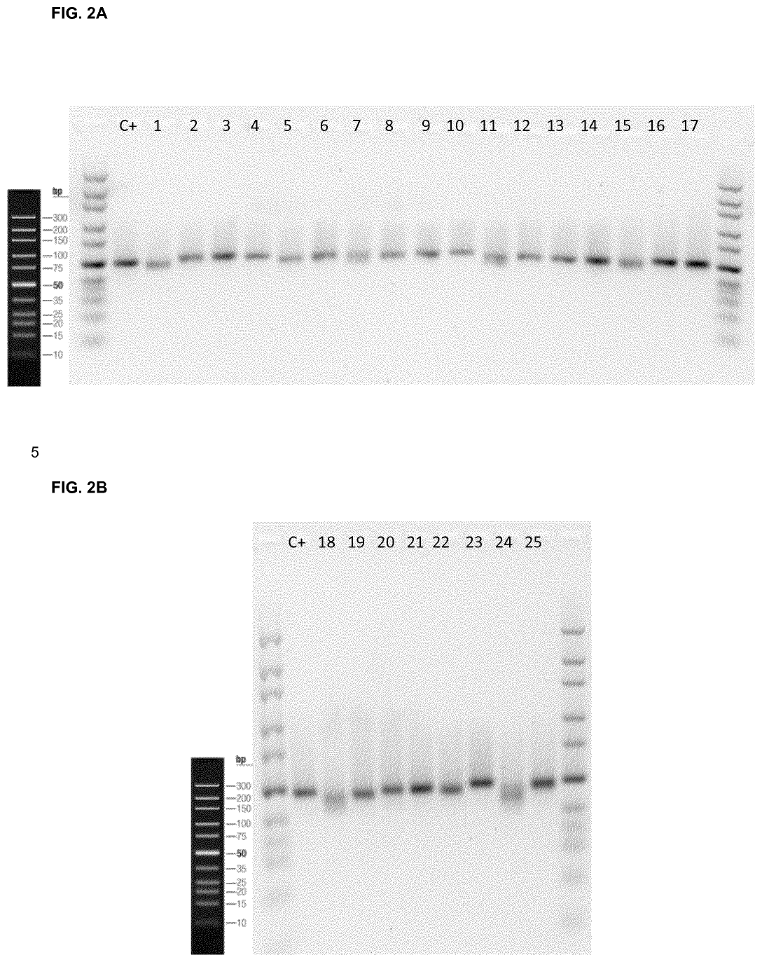

Reaction products should be checked in 3% agarose gel made by using 1× lithium borate (LB) buffer (both as casting and running buffer) pre-stained with SybrGreen (or equivalent) following the user manual (1:10 000 dilution) as shown in FIG. 2A and FIG. 2B.

In a second PCR amplification the braids were assembled.

Three μl of each of the five ropes needed for constructing a particular braid were mixed and centrifuged in separate tubes (corresponding to five different braids) according to the Table 4.

TABLE 4
Equivolume mix of ropes for obtaining the template for
constructing braids.

| | Mix of ropes to form braids | First rope | Last rope |
|---|---|---|---|
| Tube | (templates) | (string 1) | (string 2) |
| 1 | 4-1 + 1-13 + 13-12 + 12-8 + 8-10 | 4-1 | 8-10 |
| 2 | 10-11 + 11-3 + 3-1 + 1-0 + 0-7 | 10-11 | 0-7 |
| 3 | 7-3 + 3-11 + 11-10 + 10-4 + 4-0 | 7-3 | 4-0 |
| 4 | 0-5 + 5-6 + 6-3 + 3-1 + 1-9 | 0-5 | 1-9 |
| 5 | 9-4 + 4-13 + 13-2 + 2-3 + 3-5 | 9-4 | 3-5 |

Each of the five PCR reactions was performed in 20 μl volume using the following final concentrations: 200 μM of each of the dNTPs, 0.2 v/v of each of the end ropes per braid, 0.08 v/v of the mix of ropes of a particular braid and 1.2 U/100 μl reaction of Deep Vent DNA polymerase (NEB). The thermal profile was: 2 min at 95° C. (initial denaturation), followed by 35 cycles of 15 sec at 95° C., 15 sec at 55° C. and 15 sec at 72° C., ending with 5 min elongation step at 72° C.

Note that in this reaction, the mix of ropes acts as a template and it is present in the reaction in 0.08 v/v amount (1.6 μl of the rope mix per 20 μl reaction volume), whereas there are 2.5 times more of each of the two “end ropes” which act as primers, thus, are added in the reaction surplus. End ropes are the first and the last rope (Table 4) in a particular braid and each makes 20% of the reaction (0.2 v/v or 4 μl of each per 20 μl reaction).

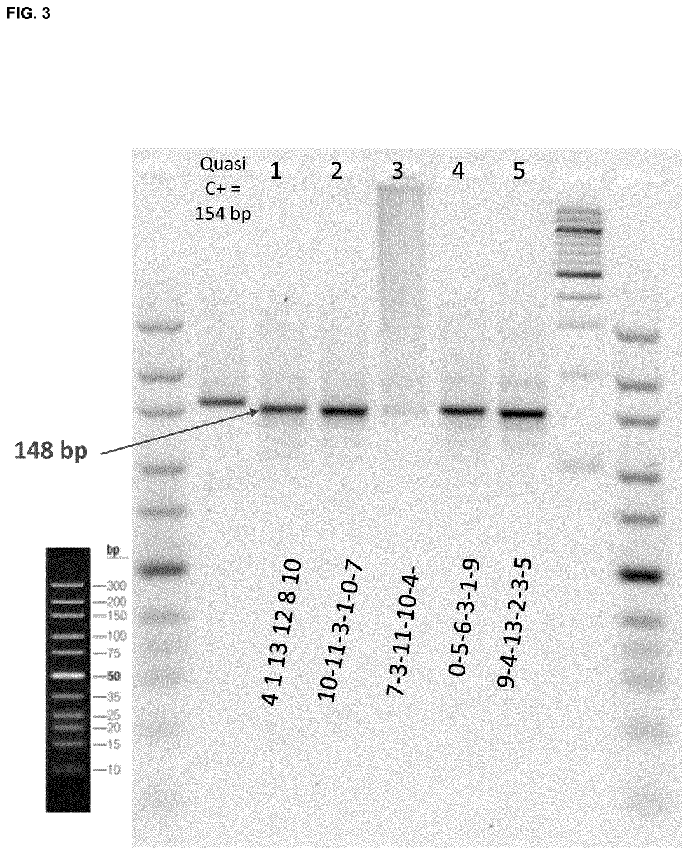

The template mix of ropes also contains end Ropes meaning that, stoichiometrically, each of the end ropes are present in the reaction in 0.2+(0.08/5)=0.216 v/v (or 21.6%). In other words, 43.2% reaction consists of the both end ropes. In that manner, the reaction is biased towards obtaining primarily the correct braid product. Reaction intermediates are present in much smaller amount, as revealed in the FIG. 3 as extra bands of the sizes greater or less than 148 bp.

In addition, no traditional purification steps are involved in this two-step assembly because they are unnecessary for the setup of the invention and due to economic reasons. Anyhow, the primers, dNTPs and enzyme that are not used in the previous reaction 1 (rope formation), are transferred and utilized in the reaction 2 (braid formation).

Prior to sequencing, braids were purified using MinElute PCR Purification Kit (Quiagen), following the user manual (membrane cut-off=70 bp−4 kb). All the centrifugations were done at 17.800×g. DNA was eluted in 20 μl H₂O which was standing for 5 minutes on the membrane prior to 2 minutes centrifugation.

To prove the correct assembly of ropes into braids, the braids were sent to a Sanger sequencing company. Primers that form 5′ and 3′ ends of a particular braid were used both to sequence that particular braid. For example, in the case of braid 1:4 1 13 12 8 10, C8G 4F and C8G 10 R were used (see Table 1). Forward primer read the last four strings (e.g. 13 12 8 10) towards the 3′ end, whereas the reverse primer read the first four strings towards the 5′ end (4 1 13 12). This was done for each of the five braids. Chromatogram data was extracted using SnapGene Viewer v. 4.1.9. All reads were readable between approx. 25-130 bases. Sequences were analyzed using EMBOSS Needle: https://www.ebi.ac.uk/Tools/psa/emboss_needle/ using default parameters.

Unspecific products were not discovered by Sanger sequencing. However, their presence might interfere with NGS technology which is thought to be utilized for the project scale-up (the size of the braids in this particular case is designed to match the 150 bp Illumina reads). This is to be easily circumvented by post-read processing ie, taking in count solely the read of the correct size (148 bp).

1. A method of storing information in nucleic acid comprising:
a) processing units of information into permutation numbers by a reversible algorithm,
b) providing a library of n distinct oligonucleotide strings of predetermined length in a fixed order, wherein n is a positive integer, wherein each distinct oligonucleotide string is associated with a distinct index indicating the ordinal position, and
c) assembling distinct oligonucleotide strings to create strands comprising at least two oligonucleotide strings,
wherein each oligonucleotide string's ordinal position matches with a permutation number, and
wherein each strand comprises at least a data bearing part and a semantic part, wherein the semantic part is to allocate orientation or order to a strand.

2. The method of claim 1 wherein the units of information are digital data.

3. The method of claim 1 wherein digital data are shortened, read as an integer, and are processed to permutation numbers by using partial permutation enumeration.

4. The method of claim 1 wherein distinct strings are assembled such that two strings are combined into a rope, and ropes are assembled to build braids.

5. The method of claim 1 wherein the library comprises two sets of distinct strings, wherein ropes are created as Cartesian products of the two sets, and wherein each rope comprises two distinct strings and a gluing part.

6. The method of claim 1 wherein the library comprises two sets of distinct strings, wherein braids are created as an alternating sequence of strings of both sets, wherein the braid a string from one of the sets is followed by a string of the other set, and wherein the sequence of the strings from each of those sets represents a partial permutation.

7. The method of claim 1 wherein assembling the strings comprises assembling braids to provide a data system comprising a head braid, at least one head/tail braid and a terminal braid, wherein in the head braid the head string is a starter string that is present only once in the first braid but at no other end of a braid in the data unit, wherein a head/tail braid comprises at least one head string, a number of center strings, and a tail string, each head string being identical to the tail string of the preceding braid thereby defining the order of the braids, and wherein in the terminal braid the tail string is a terminal string that is present only once in the terminal braid but at no other end of a braid in the data unit.

8. The method of claim 1 wherein assembling the strings comprises assembling braids comprising an ordered linear arrangement of single-stranded oligonucleotide strings selected from a library of strings, wherein the ordered linear arrangement determines the linear arrangement of a plurality of single-stranded oligonucleotide strings combined in each braid, wherein the plurality of braids comprises three types of strings, wherein one type of strings is a head string, one type of strings is a terminal string, and one type of strings is center string, wherein the single-stranded head string(s) starting from the 5′ end in the first braid is different from the single-stranded head string(s) starting from the 5′ end in any one of the other braids, and wherein the single-stranded tail string(s) starting from the 5′ end is the same as the single-stranded head string(s) starting from the 5′ end in a second braid, wherein the single-stranded tail string(s) starting from the 5′ end in the last braid of the plurality of braids is different from the single-stranded head string(s) starting from the 5′ end in any one of the other braids, and wherein the single-stranded head string(s) starting from the 5′ end is unique for each braid; and wherein the single-stranded terminal string occurs only once per data unit; and assembling the plurality of strings or ropes by PCR amplification using single-stranded strings or ropes as primers.

9. The method of claim 1 wherein the library of single-stranded oligonucleotides comprises a plurality of pairs of single-stranded oligonucleotides, wherein each pair comprises a forward and a reverse primer, wherein the forward primer comprises a first single-stranded oligonucleotide comprising a first coding sequence at its 5′ end and a first gluing sequence at its 3′ end, wherein the reverse primer comprises a second coding sequence at its 5′ end and a second gluing sequence at its 3′ end, wherein the second coding sequence is complementary and inverse to the first coding sequence, wherein the second gluing sequence is complementary and inverse to the first gluing sequence, wherein the first gluing sequence in each pair of single-stranded oligonucleotides is identical, and wherein each coding sequence is unique.

10. The method of claim 1 further comprising annealing a forward primer of a first pair of single-stranded oligonucleotide strings to a reverse primer of a second pair of single-stranded oligonucleotide strings, extending both the reverse and forward primers to obtain a double-stranded rope, wherein one strand comprises the coding sequence of the forward primer of the first pair at its 5′ end, and the coding sequence of the forward primer of the second pair at its 3′ end, wherein the gluing sequence is between the coding sequences, wherein the strings of the first and second pair have been selected to match with a permutation number obtained in a).

11. The method of claim 10 further comprising repeating the annealing until a braid has been obtained or isolating braids after assembly.

12. The method of claim 1 wherein strands are assembled by pooling double-stranded strings separately for each strand and amplifying the mixture of strings to obtain the completed strands.

13. The method of claim 1 wherein the library comprises a plurality of single-stranded ropes, each comprising two data bearing sequences, wherein the ropes have been obtained as Cartesian products of two sets of single-stranded strings, wherein optionally braids are assembled by pooling single-stranded ropes each comprising two strings in an order as defined according to a), and amplifying the mixture of ropes to obtain braids, wherein optionally a braid can be obtained by annealing ropes, wherein any rope has an overlapping part with another rope, and wherein the overlapping part is a forward primer or a reverse primer, respectively, such that one string comprises a forward primer and a second string comprises a corresponding reverse primer.

14. The method of claim 1, wherein the sequence of the single-stranded string has a length of about 4 to about 500 nucleotides, wherein the GC content is between 0.35 and 0.75; wherein the Levenshtein distance or Hamming distance between each pair of coding sequences is at least 3; wherein the number of G/C bases that are present in the last 5 to 10 bases of each coding sequence is predetermined, wherein there are up to 3 identical bases in a row, wherein the number of base repeats in each coding sequence is predetermined, and wherein a gluing sequence has a length of about 4 to about 60 nucleotides.

15. The method of claim 1 further comprising sequencing sequences of the data system and decoding the information by using the reverse algorithm.

16. A system, comprising:
a means for processing units of information into permutation numbers by a reversible algorithm,
a means for providing a library of n distinct oligonucleotide strings of predetermined length in a fixed order, wherein n is a positive integer, wherein each distinct oligonucleotide string is associated with a distinct index indicating the ordinal position,
a means for assembling distinct oligonucleotide strings to create strands comprising at least two oligonucleotide strings,
wherein each oligonucleotide string's ordinal position matches with a permutation number, and
wherein each strand comprises at least a data bearing part and a semantic part, wherein the semantic part is to allocate orientation or order to a strand.


SEQUENCE LISTING (28 sequences)

SEQ ID NO: 1 - DNA, 26 bp, Artificial Sequence
  forward primer
gcaattgatggcggtagagcgacaga

SEQ ID NO: 2 - DNA, 26 bp, Artificial Sequence
  reverse primer
tctaccgccatcaattgctctgtcgc

SEQ ID NO: 3 - DNA, 26 bp, Artificial Sequence
  forward primer
tctgcaagctggagtagagcgacaga

SEQ ID NO: 4 - DNA, 26 bp, Artificial Sequence
  reverse primer
tctactccagcttgcagatctgtcgc

SEQ ID NO: 5 - DNA, 26 bp, Artificial Sequence
  forward primer
taggatacgccacacactgcgacaga

SEQ ID NO: 6 - DNA, 26 bp, Artificial Sequence
  reverse primer
agtgtgtggcgtatcctatctgtcgc

SEQ ID NO: 7 - DNA, 26 bp, Artificial Sequence
  forward primer
taacgtcggctactcacagcgacaga

SEQ ID NO: 8 - DNA, 26 bp, Artificial Sequence
  reverse primer
tgtgagtagccgacgttatctgtcgc

SEQ ID NO: 9 - DNA, 26 bp, Artificial Sequence
  forward primer
tcgtgttagtccgtcagtgcgacaga

SEQ ID NO: 10 - DNA, 26 bp, Artificial Sequence
  reverse primer
actgacggactaacacgatctgtcgc

SEQ ID NO: 11 - DNA, 26 bp, Artificial Sequence
  forward primer
aacactcgtctcgaccatgcgacaga

SEQ ID NO: 12 - DNA, 26 bp, Artificial Sequence
  reverse primer
atggtcgagacgagtgtttctgtcgc

SEQ ID NO: 13 - DNA, 26 bp, Artificial Sequence
  forward primer
tctatgcgaccactacgagcgacaga

SEQ ID NO: 14 - DNA, 26 bp, Artificial Sequence
  reverse primer
tcgtagtggtcgcatagatctgtcgc

SEQ ID NO: 15 - DNA, 26 bp, Artificial Sequence
  forward primer
tgacagagcgtctatcgtgcgacaga

SEQ ID NO: 16 - DNA, 26 bp, Artificial Sequence
  reverse primer
acgatagacgctctgtcatctgtcgc

SEQ ID NO: 17 - DNA, 26 bp, Artificial Sequence
  forward primer
atggctatcgctgattgcgcgacaga

SEQ ID NO: 18 - DNA, 26 bp, Artificial Sequence
  reverse primer
gcaatcagcgatagccattctgtcgc

SEQ ID NO: 19 - DNA, 26 bp, Artificial Sequence
  forward primer
tcctgcgcttatcagagtgcgacaga

SEQ ID NO: 20 - DNA, 26 bp, Artificial Sequence
  reverse primer
actctgataagcgcaggatctgtcgc

SEQ ID NO: 21 - DNA, 26 bp, Artificial Sequence
  forward primer
tcgctagaagagcagagtgcgacaga

SEQ ID NO: 22 - DNA, 26 bp, Artificial Sequence
  reverse primer
actctgctcttctagcgatctgtcgc

SEQ ID NO: 23 - DNA, 26 bp, Artificial Sequence
  forward primer
agtgtccaggattgcatggcgacaga

SEQ ID NO: 24 - DNA, 26 bp, Artificial Sequence
  reverse primer
catgcaatcctggacacttctgtcgc

SEQ ID NO: 25 - DNA, 26 bp, Artificial Sequence
  forward primer
accgatacacgtcgtacagcgacaga

SEQ ID NO: 26 - DNA, 26 bp, Artificial Sequence
  reverse primer
tgtacgacgtgtatcggttctgtcgc

SEQ ID NO: 27 - DNA, 26 bp, Artificial Sequence
  forward primer
gtgcatgcatcacgatgagcgacaga

SEQ ID NO: 28 - DNA, 26 bp, Artificial Sequence
  reverse primer
tcatcgtgatgcatgcactctgtcgc